(19) United States
(12) Patent Application Publication
(10) Pub. No.: US 2020/0150004 A1
(43) Pub. Date: May 14, 2020

(54) AIR-SAMPLING KIT

(71) Applicant: The Regents of the University of California, Oakland, CA (US)

(72) Inventors: John Waldo Taylor, Berkeley, CA (US); Thomas D. Bruns, Albany, CA (US); Rachel I. Adams, El Cerrito, CA (US)

(21) Appl. No.: 16/631,827

(22) PCT Filed: Jul. 18, 2018
(86) PCT No.: PCT/US2018/042744

§ 371 Date: Jan. 16, 2020

Related U.S. Application Data
(60) Provisional application No. 62/534,577, filed on Jul. 19, 2017.

(51) Int. Cl. G01N 1/26; G01N 1/22; C12M 1/26

(52) U.S. Cl. CPC G01N 1/26; C12M 1/261; G01N 1/2273

(57) ABSTRACT

Air-sampling devices for obtaining airborne microbial samples from an environment are provided. A weathershield is also described which may be used to shield an airsampling device from the weather, moisture, animals, and other potential contaminants. These components may be provided as part of an air-sampling kit.

### CROSS-REFERENCE TO RELATED APPLICATIONS

This application claims benefit of priority under 35 U.S.C. § 119(e) to U.S. Provisional Patent Application No. 62/534,577, filed Jul. 19, 2017, which is hereby incorporated herein by reference in its entirety and for all purposes.

### TECHNICAL FIELD

The present disclosure relates to air sampling devices for collecting microbes in various indoor and outdoor environments for later analysis using various laboratory techniques, e.g., PCR amplification and DNA sequencing, to allow for later characterization of the microbial community in the sampled environment.

### BACKGROUND

Poorly maintained buildings (and even well-maintained buildings, on occasion) may frequently contain microbial communities that may be potentially harmful to the occupants, or to a subset of particularly vulnerable occupants, e.g., asthma sufferers or people with allergic responses to certain microbes. Testing for microbes may therefore be of interest in order to characterize such buildings and the potential health effects that the occupants thereof may experience due to exposure to such microbial communities and to identify buildings or rooms therein that require maintenance to deal with various forms of water intrusion or other potential sources of microbial life.

### SUMMARY

Discussed herein are various implementations of air-sampling devices that facilitate collection of microbial samples in a manner that allows for later characterization of the microbial community as a whole, e.g., in a manner that preserves the ability to determine the presence and approximate concentrations of various types of microbes in the microbial community. Such air-sampling devices may be used to sample air that is within a structure as well as air that is immediately outside of the structure, e.g., ambient outdoor air, so that a differential analysis on the microbial communities may be performed to determine how the microbial community within the structure differs from the microbial community outside of the structure.

In some implementations, an air-sampling kit is provided that includes a) two or more air-sampling devices and b) a weathershield. Each air-sampling device of such a kit may include an upper half and a lower half, and the upper half and the lower half of the air-sampling device may be joined by a hinge device configured to permit relative rotation between the upper half and the lower half about at least a first hinge axis, the upper half and the lower half may nestle together when closed to provide an enclosure, the surfaces of the upper half and the lower half that form the boundaries of the enclosure when the upper half and the lower half area nestled together may be sterile, and the upper half and the lower half may be rectangular. The air-sampling devices may not only be sterile, but may also not include any culture medium. The weathershield may include a floor, two side walls, and a ceiling. The side walls may extend upwards from the floor and the ceiling may span between the side walls such that the sidewalls, the ceiling, and the floor form a tunnel with a first end and a second end. A first air-sampling device of the two or more air-sampling devices may be configured to be insertable into the tunnel and rest on the floor, and the side walls and the ceiling may extend beyond the floor along an axis extending from the first end to the second end by a first distance on both sides of the floor.

In some such implementations, the first distance may be at least 100% to 110% of the width of the first air-sampling device along a direction parallel to the first axis of the first air-sampling device.

In some implementations of the kit, the weathershield may be made from a transparent material.

In some implementations of the kit, the kit may also include a plurality of suction cups located on one of the side walls.

In some implementations of the kit, the air-sampling devices may all be the same size and shape.

In some implementations of the kit, the ceiling may be curved, forming an arch with the side walls.

In some implementations of the kit, the ceiling may be sloped towards one of the side walls.

In some implementations of the kit, the air-sampling devices may be sterile, square petri dishes with no culture medium.

In some implementations of the kit, the weathershield may have mesh screens screening off the first end and second end.

In some implementations of the kit, the weathershield is flattenable.

In some implementations of the kit, the first air-sampling device and the weathershield may have corresponding mechanical interfaces that are configured to interconnect and secure the first air-sampling device to the weathershield.

In some implementations of the kit, the corresponding mechanical interfaces may be hook-and-loop fasteners.

In some implementations of the kit, each air-sampling device may have a closure mechanism configured to restrain the upper half and the lower half of that air-sampling device from opening.

The above is not an exhaustive list of all of the aspects of the concepts discussed herein, and further aspects of the disclosure will become apparent from the Figures and the discussion below.

### BRIEF DESCRIPTION OF THE DRAWINGS

The present disclosure is illustrated by way of example, and not limitation, in the figures of the accompanying drawings, in which like reference numerals indicate similar elements unless otherwise indicated.

FIG. 1 is a photograph of an example air-sampling device kit that includes an example weathershield.

FIG. 2 is a drawing showing an example air-sampling device similar to the air-sampling device in the kit of FIG. 1 in various stages of use.

FIG. 3 is drawing of an example air-sampling device kit similar to that shown in FIG. 1.

FIG. 4 depicts two different cross-sectional shapes for a weathershield.

FIG. 5 is a photograph of the example weathershield of FIG. 1 and an example air-sampling device installed on a window.

FIG. 6 depicts an example weathershield with a mesh screen installed.

FIG. 7A is a diagram of an example weathershield and air-sampling device.

FIG. 7B is a diagram of the example weathershield in a collapsed state.

### DETAILED DESCRIPTION

Importantly, the concepts discussed herein are not limited to any single aspect or implementation discussed herein, nor to any combinations and/or permutations of such aspects and/or implementations. Moreover, each of the aspects of the present invention, and/or implementations thereof, may be employed alone or in combination with one or more of the other aspects and/or implementations thereof. For the sake of brevity, many of those permutations and combinations will not be discussed and/or illustrated separately herein.

As discussed above, the present disclosure provides for a variety of air-sampling devices, as well as techniques for using and creating them, that may be used to more effectively sample air within a structure or structures in order to characterize the microbial communities within such structures.

Such air-sampling devices were developed to allow for post-sampling characterization of the microbial communities in the sampled environments in a manner that preserves the presence and relative concentrations of the microbes that form the microbial community. Another aim of the air-sampling devices was to allow for corresponding samples to be collected simultaneously from different locations in a structure as well as from the outside ambient environment to facilitate performing characterizations of the various locations in the structure and normalizing the samples to account for the contributions to the microbial community that are simply due to the ambient outside environment as opposed to microbes arising due to an issue, e.g., a mold colony, within the building. A further aim of some implementations of such air-sampling devices was to allow for unobtrusive sample collection, particularly with respect to the collection of samples from locations outside of the building in question.

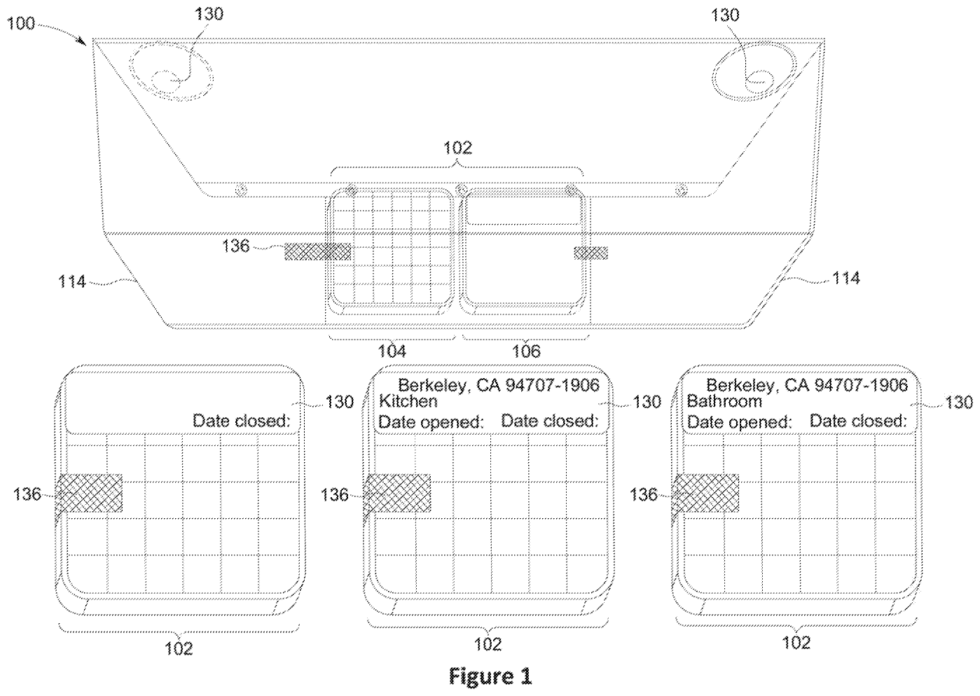

FIG. 1 is a photograph of an example air-sampling device kit, which includes an example weathershield, in accordance with the present disclosure. The air-sampling device kit 100 includes four air-sampling devices 102 and a weathershield 114. The weathershield 114 that is depicted in FIG. 1 has one of the air-sampling devices 102 in a deployed or open state located within it; the deployed air-sampling device 102 has, in this case, an upper half 106 and a lower half 104. The air-sampling devices 102 located in the foreground of FIG. 1 are similarly sized and configured to the air-sampling device 102 located within the weathershield 114, although these foreground air-sampling devices 102 are in a closed state, i.e., not deployed for air-sampling. Each of the air-sampling devices 102 may be equipped with a label 138 that may include, for example, locations for annotations describing the location in which that air-sampling device 102 was deployed/located, the address (including apartment or unit number, if desired) of the domicile, office, or other building/rooms at which it was deployed, and the dates and, optionally, times at which it was initially deployed and then later retrieved. The weathershield 114, as can be seen, includes features, such as suction cups 130, to allow it to be mounted to a window or other flat, impermeable exterior surface.

In various implementations, the air-sampling device 102 may be a sterile, closable container, preferably one that is easy to open and close without causing the interior surfaces of the container to come into contact with any solids or liquids, e.g., without coming into contact with the user's hand. The air-sampling device 102 may also preferably be rectangular or square in aspect ratio (when laid flat) so as to maximize sampling area within a given rectangular area. In testing, square plastic petri dishes, such as the clear polystyrene sterile square petri dishes sold by SKS Science Products and shown in FIG. 1, were found to provide good air-sampling performance. Such petri dishes, for example, may be approximately 90 mm square and 15 mm deep. The petri dishes may generally include two complementary halves, each including a base area (e.g., 90 mm square) that has a peripheral wall extending upwards from the base area, e.g., 15 mm high. The two complementary halves may be sized such that one half (the upper half 106) is a lid for the other half (the lower half 104), e.g., the peripheral walls of the “lid” half may have an inner perimeter that is sized just slightly larger than the outer perimeter of the peripheral walls of the other half. The thickness of the base and walls may be on the order of a millimeter or so.

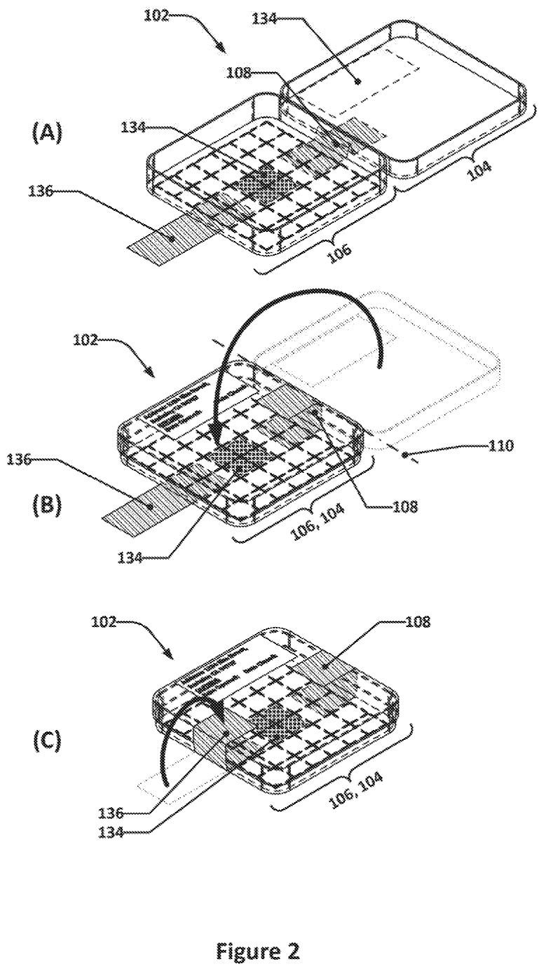

FIG. 2 depicts an example air-sampling device 102 in a deployed state (A), a closed state (B), and a closed and secured state (C). As can be seen in (A), in the deployed state, the upper half 106 and the lower half 104 of the air-sampling device 102 are positioned adjacent to one another with their interior surfaces facing upwards and exposed to the air. The lower half 104 and the upper half 106 may be connected to one another by a hinge device 108, which, in this example, is a hinge that is provided by a flexible strip of material, e.g., tape, that is adhered to the lower half 104 and the upper half 106 to allow the upper half 106 and the lower half 104 to be hinged open (or closed) about a hinge axis 110 (note that for a hinge device that is rigid, such a hinge axis may be substantially fixed in place relative to the two halves, but in the depicted example, with a flexible hinge, the hinge axis 110 may move about somewhat due to flexure in the hinge device itself. After air-sampling has been conducted with the deployed air-sampling device 102 as shown in (A), the upper half 106 may be rotated about the hinge axis 110 towards the lower half 104 until the air-sampling device 102 is in the configuration shown in (B), i.e., closed (the dotted outline represents where the upper half 106 was prior to such closure). Once closed, whatever microbes have been collected within the air-sampling device 102 will generally be trapped there until the air-sampling device is opened, e.g., in a laboratory. To prevent inadvertent re-opening of the air-sampling device 102, a close mechanism 130 may be provided to allow the upper half 106 to be secured to the lower half 104. The closure mechanism 130, in this case, is a piece of tape, although other types of closure mechanisms may be used as well, e.g., a magnet, a latch, a rubber band, or any of a variety of other devices. The hinge device 108 may also prevent the two halves of the air-sampling device 102 from separating and becoming mixed up with a similar half of one of the other air-sampling devices 102 in the kit.

The air-sampling device 102 shown in FIG. 2 also includes a label 138, which indicates that, in this case, the air-sampling device 102 was located outside, i.e., in the ambient outside environment, e.g., within a weathershield 114. The air-sampling device 102, in this instance, also includes a mechanical interface 134, e.g., a hook-and-loop fastener, for securing the air-sampling device 102 within the weathershield 114. Air-sampling devices 102 that are not intended to be mounted in a weathershield 114 may omit such mechanical interfaces 134.

It is to be understood that such “sterile” containers, as the term is used herein, are containers that are not only sterile but also devoid of biological nutrients or culture media, e.g., agar. Thus, the containers may be, for example, glass or plastic that has been sterilized (at least with regard to the interior surfaces) and that is otherwise empty of culture media that may promote or facilitate microbial growth. The aim is to capture microbes in the container without facilitating their ability to grow and reproduce.

In order to prevent the containers from undesirably opening before or after collection and before being opened for laboratory analysis, and to prevent the halves from two different containers from being accidentally mismatched or separated, the two halves of each such container may be connected together with a hinge that may allow the containers to be easily opened. In experiments performed with the square petri dish air samplers discussed above, a simply hinge was constructed using tape to create a flexible hinge. The containers may also include some form of fastening device or other closure mechanism that may be used to prevent the containers from opening at all until the fastening device or closure mechanism is removed or disengaged. For example, the fastening device or closure mechanism may be a magnet, tape, a latch, a rubber band, or any of a variety of other devices.

The sterility of the air-sampling container is important, as it is undesirable to have a medium in the air-sampling container that might prove hospitable to microbial growth. For example, if the petri dish were to contain agar, the microbes that are collected by that petri dish would start to grow and multiply, with the more aggressive species rapidly overwhelming the less aggressive species. In relatively short order, this growth would cause the microbial population in the air-sampling device to have microbial concentrations that deviate, likely in a significant manner, from the microbial concentrations in the microbial population at the sampling location. Such imbalances would result in incorrect characterization of the sampled environments, thereby rendering the results useless.

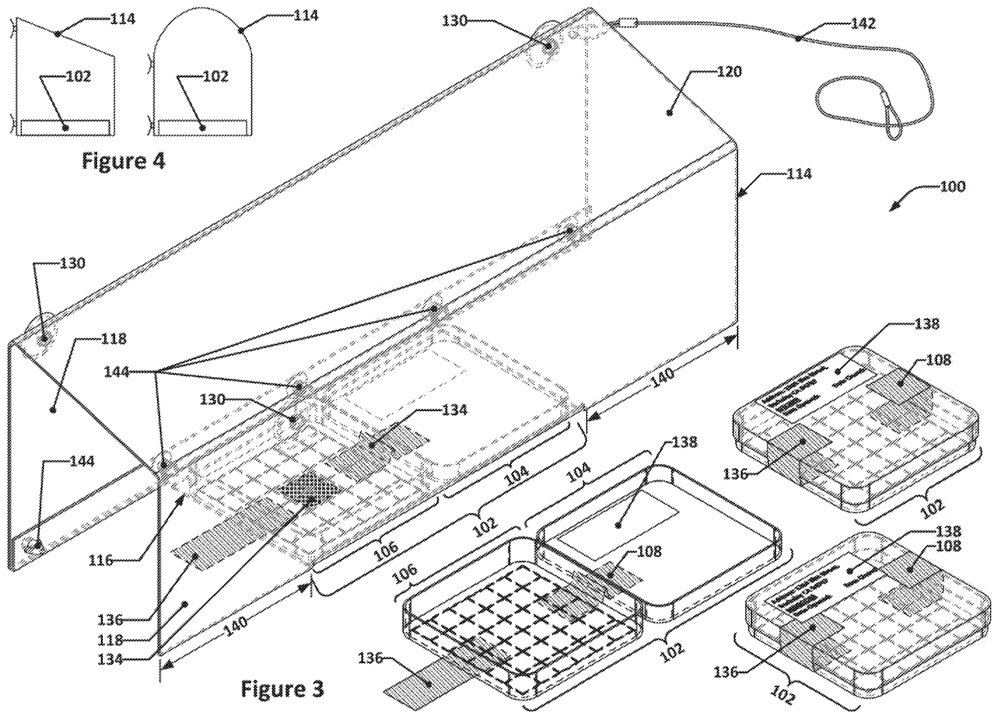

In order to characterize multiple locations, as well as to obtain a baseline ambient outdoor environment microbial characterization, sampling kits may be used that contain multiple air-sampling devices, as well as at least one weathershield device configured to receive one of the air-sampling devices and to be mounted or secured in an outdoor environment. An example such sampling kit is depicted in FIG. 3, which shows an air-sampling kit similar to that shown in FIG. 1.

The air-sampling devices 102 within the kit 100 are generally the same size and type so that each presents a generally equivalent sampling opportunity, i.e., the concentrations of a microbe determined from each sample may generally be assumed to be to-scale between each sample (assuming that both samples were collected during the same durations of time). For example, if an air-sampling device collects microbial samples indicating that a particular microbe is present in a concentration that is twice as high as that of another air-sampling device, then it may be assumed that the concentration of that microbe in the sampling location of the first air-sampling device is approximately twice as high as it is in the sampling location of the second air-sampling device.

Air-sampling devices 102 that are intended to be used in indoor environments may be used without any special accessories, and may be placed in any suitable location. Preferably, such locations may be locations such as on top of countertops, dressers, or other locations not directly on the floor.

The air-sampling device 102 (or devices) intended for outdoor use, however, require the use of a weathershield 114 in many cases, as the air-sampling device 102 may need to be protected from moisture, snow, animals, etc.

The weathershield 114 may, for example, take the form of a tunnel that is open on both ends. The tunnel may have a floor 116 that is configured to support an air-sampling device 102 when the air-sampling device 102 is installed in the weathershield 114, as well as a ceiling 120 and sidewalls 118. The ceiling 120 and sidewalls 118 may extend past the edges of the floor 116 along the long axis of the tunnel, thereby avoiding the presence of horizontal surfaces near the air-sampling device and within the tunnel near the opposing ends of the tunnel. Such horizontal surfaces, were they to be present, would provide a resting ledge for rain or other moisture to collect on, thereby increasing the chance of pooling water and moisture contamination of the air-sampling device 102. The absence of such horizontal surfaces near the ends of the tunnel may also make it more difficult for animals, such as birds, to enter the tunnel. The ceiling of the tunnel may be sloped or curved to as to prevent water from pooling on the upper surface of the ceiling (or to prevent snow from collecting on it), thereby also reducing the chance of moisture contamination. For example, the weathershield 114 may, as shown at left in FIG. 4, have a sloped ceiling 120 or may, as shown at right in FIG. 4, have a curved ceiling 120 (both views are end views, e.g., looking along the long axis of the weathershield 114).

In some implementations, the ceiling 120 and the side walls 118 may extend along the longitudinal axis of the weathershield 114 beyond the transverse edges of the floor 116 by a first distance 140, i.e., the edges of the floor may be inset along the longitudinal axis from the outermost edges of the ceiling 120 and the side walls 118 by the first distance 140 when viewed along an axis perpendicular to the air-sampling device 102, e.g., the vertical axis. The first distance 140 may, for example, be at least 100% to 110% of the width of the air-sampling device 102 (in the closed state—in the open state, the first distance 140 may be at least 100% to 110% of the width of either the upper or lower half of the air-sampling device).

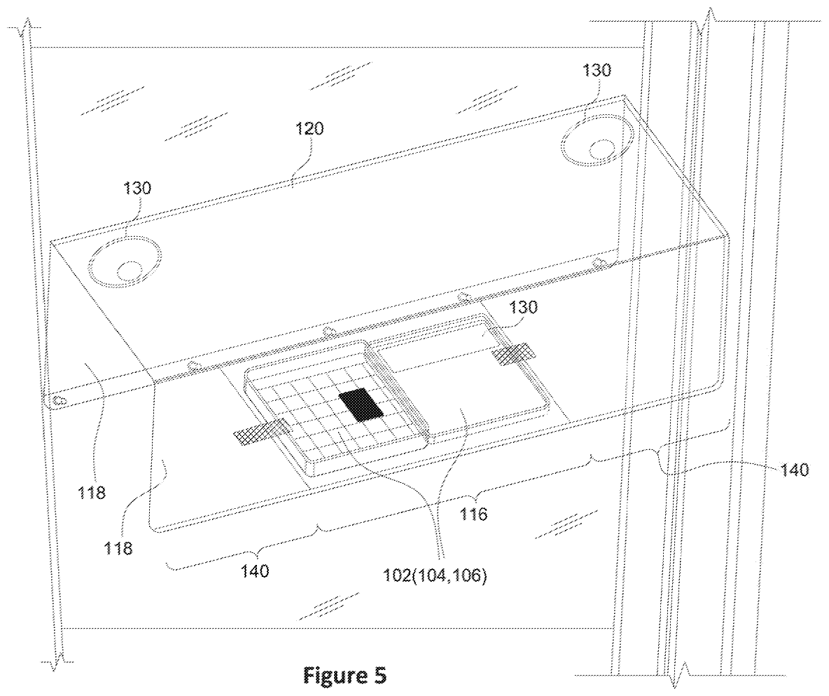

The weathershield 114 may have features that allow it to be mounted to an external wall or window of a building, thereby allowing for easy installation. For example, in some implementations, the weathershield 114 may include a plurality of suction cups 130 that may be used to affix the weathershield 114 to a window, as is shown in FIG. 5, which is a photograph of the weathershield 114 of FIG. 1 mounted to a window. Such an implementation may be particularly useful for characterizing apartments, as such locations often have limited access to outdoor locations, and may even have no porches, decks, yards, etc. in which to place the weathershield 114—windows may therefore be the only viable location. Moreover, even if access to a ground-based location, e.g., a yard, were to be available for an apartment, it would still be desirable to obtain the ambient outdoor sample at the same elevation as the interior samples are obtained to sample the microbial population of the ambient outdoor environment most similar to that likely to enter the apartment or condominium from outside.

While the weathershield may be made of a variety of materials, plastics and other non-permeable materials may generally be preferable, as they are cheap, weather resistant, lightweight, and easily shaped. While metal weathershields may also be used, such structures will be more expensive and heavier, which may present an increased risk of injury should one fall from an elevated location. In some implementations, it may be preferable to make the weathershield out of a clear or transparent material, such as transparent polycarbonate. This serves several purposes: a) it allows for visual inspection of the air-sampling device from within the building when the weathershield and air-sampling device are mounted to a window, b) it reduces the amount of light that is blocked by the weathershield, thereby preserving the natural illumination within the building, and c) it makes the weathershield and air-sampling device much more difficult to see from outside the building, especially when used at elevated locations. This last factor may be important since building owners or managers may wish to discourage air-sampling within their buildings, as the results may be used to potentially force them to take remedial action to address potential microbial contamination issues, which may be expensive. If the air-sampling may be conducted in relative secrecy, however, this may permit tenants of such buildings to determine if there are any microbial issues that may be affecting their health without necessarily alerting the landlord or building owner, thereby allowing for collection of the sample to occur without risk of interference.

The weathershield 114 and/or ambient outdoor air-sampling device 102 may also include features designed to secure the air-sampling device 102 in the weathershield 114. For example, such an air-sampling device 102 may be equipped with a mechanical interface 134, e.g., a piece of hook-and-loop fastener system that is affixed to the lower half, and the floor 116 of the weathershield 114 may have a corresponding piece of the mechanical interface, e.g., a corresponding piece of the hook-and-loop fastener system, to allow the air-sampling device 102 to be attached to the floor 116. In some implementations, the weathershield and/or ambient outdoor air-sampling device may also include a safety tether or lanyard 142 that may be attached to either or both devices and then secured to either a feature on the building or inside the building. This may prevent injury to passers-by below if the unit should detach from its mounting location.

As discussed, each air-sampling device 102 may be pre-labeled with an identifier on the outside to allow that air-sampling device 102 to be tracked. The identifier may, for example, be an adhesive label 138 that identifies what room the air-sampling device was (or is intended to be) placed in, e.g., “kitchen,” “living room,” “bedroom 1,” “bedroom 2,” etc. One of the air-sampling devices may be labeled as an “outside” air-sampling device 102; this particular air-sampling device 102 may be designed to be placed in the weathershield 114 (and may thus, for example, have extra features to attachment to the weathershield 114).

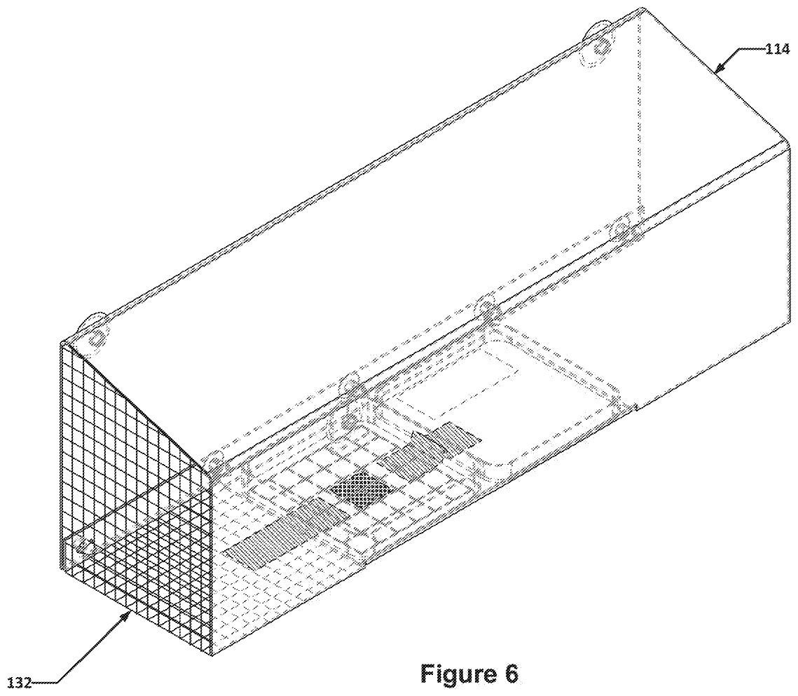

The tunnel aspect of the weathershield 114 may allow air to blow through the weathershield and across the air-sampling device contained within. In some implementations, the open ends of the weathershield may have mesh screens or barriers to allow air to blow through but keep out birds and insects, which may contaminate the air-sampling device 102; FIG. 6 depicts an example weathershield 114 with a mesh screen 132 placed across one open end of the tunnel; a similar mesh screen would be placed across the other end of the tunnel as well, although the additional mesh screen is not shown in this view.

In practice, the components of the air-sampling kit may be packaged together in a common box (or even an envelope, if the weathershield is collapsible) and mailed to an interested party. The air-sampling devices within may be distributed to various locations within the structure or building in which sampling is to occur, with at least one air-sampling device being installed in the weathershield. The locations (and installation dates and times) of the air-sampling devices may be recorded, e.g., on labels on each air-sampling device, and the weathershield (and installed air-sampling device) may be installed outside, e.g., on a window. At each sampling location, the air-sampling devices may be opened so that the sterile interiors are exposed to the air in that sampling location. After a suitably long enough sampling period, e.g., a week, the air-sampling devices may be carefully closed up again (taking care not to contaminate the interiors of the air-sampling devices), fastened shut, and then mailed to an analysis center, where sterile swabs or the like may be used to swab the interiors of the air-sampling units to transfer the collected microbes to an analysis system for characterization.

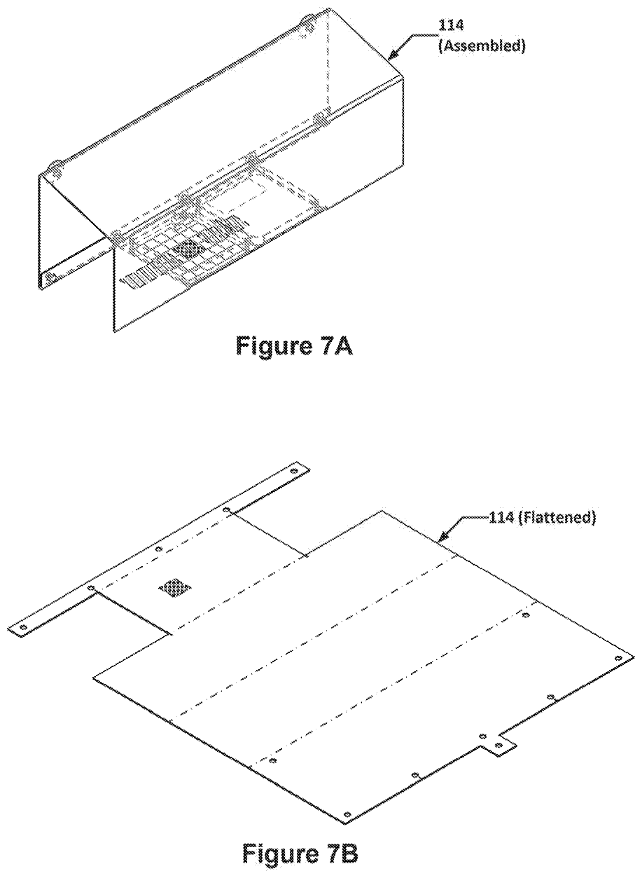

As mentioned above, the weathershield 114 may be collapsible. FIGS. 7A and 7B depict an example weathershield in the uncollapsed state (FIG. 7A) with air-sampling device 102 positioned within) and in a collapsed state (FIG. 7B). The flattened or collapsed weathershield 114 may be mailed in an envelope (in some instances, the weathershield may be partially folded, e.g., folded in half, to allow it to fit within a smaller envelope or package) and then folded along the dash-dot-dash lines (which may, in some cases, be pre-formed creases or actual printed lines) to form a trapezoidal cross-section structure (or other weathershield shape); the mating ends of the weathershield 114 may be joined together, for example, with snaps 144 (see FIG. 3) or other fastening means. In some implementations, the narrow strips extending away from the floor 116 where the snaps 144 are located may be shortened in length or entirely omitted, e.g., only a length of material as long as the floor 116 may overlap with the sidewall 118. Suction cups or other support devices may be added to the weathershield as well.

Various articles to which the inventors contributed may provide additional context regarding passive microbe collection and analysis and are incorporated by reference herein for all that they disclose. These articles include:

“Passive dust collectors for assessing airborne microbial material” by Rachel I. Adams, Yilin Tian, John W. Taylor, Thomas D. Bruns, Anne Hyvarinen, and Martin Täubel; published in Microbiome (2015) 3:46.

“Chamber Bioaerosol Study: Outdoor Air and Human Occupants as Sources of Indoor Airborne Microbes” by Rachel I. Adams, Seema Bhangar, Wilmer Pasut, Edward A. Arens, John W. Taylor, Steven E. Lindow, William W. Nazaroff, and Thomas D. Bruns; published in PLOS ONE 10(7): e0133221 in 2015.

“The Diversity and Distribution of Fungi on Residential Surfaces” by Rachel I. Adams, Marzia Miletto, John W. Taylor, and Thomas D. Bruns; published in PLOS ONE 8(11): e78866 in 2013.

“Dispersal in microbes: fungi in indoor air are dominated by outdoor air and show dispersal limitation at short distances” by Rachel I. Adams, Marzia Miletto, John W. Taylor, and Thomas D. Bruns; published in The ISME Journal 1-12 in 2013.

“A Unique Signal Distorts the Perception of Species Richness and Composition in High-Throughput Sequencing Surveys of Microbial Communities: a Case Study of Fungi in Indoor Dust” by Rachel I. Adams, Anthony S. Amend, John W. Taylor, and Thomas D. Bruns; published in Microbial Ecology 66(4): 735-41 in November 2013.

“Airborne Bacterial Communities in Residences: Similarities and Differences with Fungi” by Rachel I. Adams, Marzia Miletto, Stephen E. Lindow, John W. Taylor, and Thomas D. Bruns; published in PLOS ONE 9(3): e91283 in 2014.

All six of the above references are hereby incorporated herein by reference in their entireties.

It is to be understood that the above disclosure, while focusing on a particular example implementation or implementations, is not limited to only the discussed example, but may also apply to similar variants and mechanisms as well, and such similar variants and mechanisms are also considered to be within the scope of this disclosure.

1. An air-sampling kit comprising:
a) two or more air-sampling devices, wherein, for each air-sampling device:
the air-sampling device includes an upper half and a lower half,
the upper half and the lower half of the air-sampling device are joined by a hinge device configured to permit relative rotation between the upper half and the lower half about at least a first hinge axis,
the upper half and the lower half nestle together when closed to provide boundaries that define an enclosure,
the surfaces of the upper half and the lower half that form the boundaries of the enclosure when the upper half and the lower half are nestled together are sterile, and
the air-sampling device does not include any culture medium, and
b) a weathershield, the weathershield including:
a floor,
two side walls, and
a ceiling, wherein:
the side walls extend upwards from the floor and the ceiling spans between the side walls,
the sidewalls, the ceiling, and the floor form a tunnel with a first end and a second end,
a first air-sampling device of the two or more air-sampling devices is configured to be insertable into the tunnel and to rest on the floor, and
the side walls and the ceiling extend beyond the floor along an axis extending from the first end to the second end by a first distance on both sides of the floor.

2. The air-sampling kit of claim 1, wherein the first distance is at least 100% to 110% of the width of the upper half of the first air-sampling device along a direction perpendicular to the first hinge axis of the first air-sampling device.

3. The air-sampling kit of claim 1, wherein the weathershield is made from a transparent material.

4. The air-sampling kit of claim 1, further comprising a plurality of suction cups located on one of the side walls.

5. The air-sampling kit of claim 1, wherein the air-sampling devices are the same size and shape.

6. The air-sampling kit of claim 1, wherein the ceiling is curved, forming an arch with the side walls.

7. The air-sampling kit of claim 1, wherein the ceiling is sloped towards one of the side walls.

8. The air-sampling kit of claim 1, wherein the air-sampling devices are sterile, square petri dishes with no culture medium.

9. The air-sampling kit of claim 1, wherein the weathershield has mesh screens screening off the first end and second end.

10. The air-sampling kit of claim 1, wherein the weathershield is flattenable.

11. The air-sampling kit of claim 1, wherein the first air-sampling device and the weathershield have corresponding mechanical interfaces that are configured to interconnect and secure the first air-sampling device to the weathershield.

12. The air-sampling kit of claim 11, wherein the corresponding mechanical interfaces are hook-and-loop fasteners.

13. The air-sampling kit of claim 1, wherein each air-sampling device has a closure mechanism configured to restrain the upper half and the lower half of that air-sampling device from opening.

14. The air-sampling kit of claim 1, wherein the hinge device for at least one of the two or more air-sampling devices is a flexible hinge.

15. The air-sampling kit of claim 1, wherein, for at least the first air-sampling device of the two or more air-sampling devices, the upper half and the lower half are rectangular.

16. The air-sampling kit of claim 1, wherein:
a first side wall of the two side walls is higher than a second side wall of the two side walls, thereby causing the ceiling to slope downwards from the first side wall to the second side wall; and
the first side wall has a plurality of suction cups located thereupon.

17. The air-sampling kit of claim 1, further comprising a container selected from the group consisting of: an envelope or a box, wherein:
the two or more air-sampling devices and the weathershield are packaged together in the box.

18. The air-sampling kit of claim 17, wherein the weathershield is flattenable and the weathershield is packaged in the envelope or box in a flattened state.
